(19) United States
(12) Patent Application Publication
(10) Pub. No.: US 2006/0084913 A1
(43) Pub. Date: Apr. 20, 2006

(54) Safety syringe with needle orienting mechanism

(76) Inventor: Jui-Chung Lo, Sijhih City (TW)

Correspondence Address: Yi-Wen Tseng, 4331 Stevens Battle Lane, Fairfax, VA, 22033, US

(21) Appl. No.: 10/883,701

(22) Filed: Jul. 6, 2004

(51) Int. Cl. A61M 5/00; A61M 5/31

(52) U.S. Cl. 604/110; 604/240

(57) ABSTRACT

A safety syringe with a needle orienting mechanism is capable of securing the needle during normal utilization, disconnecting the needle from orientation after utilization, and drawing the needle back into the safety syringe for safety discarding. The safety syringe has a syringe barrel, a plunger, and a needle assembly. By wedging the retainer buttons of the syringe barrel into the retainer button sockets of the needle assembly, the needle is oriented firmly at its designated location during normal utilization. A drawing mechanism of the plunger is capable of releasing the needle assembly from orientation by rotating the plunger, and drawing the needle assembly back into the syringe barrel. An interlocking mechanism of the needle assembly corresponding to the drawing mechanism is disposed at the rear end of the needle assembly. The drawing mechanism has an anchor shaped reverse hook extended from and formed at the rear end of the piston thereof. The anchor shaped reverse hook is capable of being inserted into the interlocking mechanism and proceeding the interlocking and the drawing back of the needle assembly.

### BACKGROUND OF THE INVENTION

The present invention relates in general to a safety syringe, and more particularly, to a safety syringe with a needle orienting mechanism, which is capable of securing the needle during normal utilization, disconnecting the needle from orientation after utilization, and drawing the needle back into the safety syringe for safety discarding.

Since the time when medical science invented the syringe for the purpose of delivering drugs into a patient, the syringe has remained one of fastest and most effective drug delivery methods. Usage of the syringe has been widely accepted among those in the medical community. To satisfy the safety and sanitary requirements of medical facilities, hospitals mostly employ disposable syringes, and therefore, produce more dangerous medical waste. Because the syringe needle is exposed at the end of the syringe, it is possible that medical personnel or refuse collection personnel could be pricked by an exposed needle and infected when they attempt to put the protective needle cover over the needle. Consequently, a serious problem is how to reduce the risk of possible infection from the exposed needle. To solve this problem, manufacturers continuously research, improve, and provide various types of safety syringes. Among those safety syringes, the so-called draw back type safety syringe is the most popular among users.

To use the so-called safety syringe medical personnel push the plunger toward the top of the syringe which is capable of interlocking with the needle housing. By utilizing this interlocking, medical personnel can pull the needle and its housing back into the syringe, and therefore, reduce the risk that a medical or refuse worker makes direct contact with the exposed syringe needle. The typical structure of a draw back type safety syringe includes a plunger, which can be pushed forward or pulled backward within the syringe barrel; a piston installed at the end of the plunger, with a hook portion protruding from the end of the piston and connecting to the piston via a neck column; an interlocking receptacle located at the bottom of the needle. When the plunger is pushed toward the top-most point, the interlocking hook can be inserted into the interlocking receptacle to form a hook receptacle combination. When the plunger is thereafter pulled backward, the needle can then be pulled and stored within the syringe barrel.

Certainly, the aforementioned is only one draw back structure of the draw back type syringe. According to the disclosure of known patents or the formerly mentioned patent, there are different draw back structure types, for example, the interlocking receptacle located at the circumference of the needle bottom while the interlocking hook of the piston is correspondingly installed on the circumference thereof.

However, no matter which structure the draw back syringe employs, the combination of the interlocking hook and the interlocking receptacle utilizes the forward pressure on the piston to complete a “plug in” style combination. Once the interlocking hook and the interlocking receptacle are engaged together, it is not easy to release their combination in order to disconnect them. But during transportation or operation incompetence or error could push the plunger forward and cause an error combination. The syringe in the case of an error combination would become useless and would have to be abandoned. These error combinations not only are needless wastes of money, but also increase the disposal treatment fees. Furthermore, in the aforementioned already known draw back type syringes, the needles thereof are merely plugged and secured into the rear ends of the syringe barrels. There are not any orienting structures whatsoever. Therefore, the syringe needles could become disengaged inadvertently by medical personnel. For example, for most of the syringe usages, medical personnel hold the syringe and stick the needle directly into a vial in order to store an amount of the medication inside of the syringe barrel. Because the top of the vial is made from a resilient rubber material seal element, a large amount of force must be applied in order to pierce this seal element. This applied force in turn will apply an anti-reaction force onto the needle; thereby, the disengagement of the syringe needle occurs frequently. In sum, the sleeve clasp combination of the aforementioned syringe needles does not provide a suitable orienting effect; therefore, it is frequent that the needle falls into the syringe barrel while medical personnel are attempting to pierce the top seal of a vial. Certainly, the syringe cannot be utilized to inject the drug after the needle has fallen into the syringe barrel. It is because of the fallen needle that the syringe will cost the medical facility not only to replace it, but also to discard it.

### BRIEF SUMMARY OF THE INVENTION

The present invention provides a safety syringe with an orienting mechanism, which is capable of remedying the aforementioned conventional drawbacks: (a) the error combinations caused by the “plug in” style combination of the interlocking hook and the interlocking receptacle, and (b) the needle falling problem while medical personnel are attempting to pierce the top seal of a vial.

The safety syringe provides by the present invention has a pair of retainer buttons formed oppositely on the interior of the syringe barrel. By utilizing the combination of the retainer buttons and the orienting mechanism, the needle assembly is securely oriented inside the syringe barrel so that the needle falling problem is solved by the present invention.

The safety syringe provides by the present invention has an interlocking mechanism in the needle assembly main body, and a drawing mechanism at the rear end of the plunger. The plunger can be interlocked with the needle assembly by inserting the drawing mechanism into the interlocking receptacle of the needle assembly and further rotating the plunger 90 degrees to hide the anchor shaped reverse hook of the drawing mechanism within the needle assembly. This combination of the plunger and the needle assembly is a rotational interlocking; therefore, error combination is effectively reduced, and is easy to disconnect. The needless wastes of money caused by error combination and the disposal treatment fees of the abandoned syringes are therefore reduced.

The safety syringe of the present invention further has a hook disconnecting guide notch. By rotating the plunger, the anchor shaped reverse hook is capable of pressing against the vertical retaining tabs and further driving the needle assembly to rotate synchronously. The retainer buttons will thereby be disengaged from the retainer button sockets of the needle assembly, and slid into the hook disconnecting guide notch. The needle assembly is therefore released from orientation. Because the retainer buttons have been slid into the hook disconnecting guide notch, the needle assembly is capable of being further pulled back into the syringe barrel.

These and other objectives of the present invention will become obvious to those of ordinary skill in the art after reading the following detailed description of preferred embodiments.

It is to be understood that both the foregoing general description and the following detailed description are exemplary, and are intended to provide further explanation of the invention as claimed.

### BRIEF DESCRIPTION OF THE DRAWINGS

These as well as other features of the present invention will become more apparent upon reference to the drawings therein:

FIGS. 1A and 1B are different vertical cross sectional views of a safety syringe of the present invention.

FIGS. 2A and 2B are different front views of the plunger of a safety syringe of the present invention.

FIG. 3 is a vertical cross sectional view of the syringe barrel of a safety syringe of the present invention.

FIGS. 4A and 4B are different vertical cross sectional views of the needle assembly plunger of a safety syringe of the present invention.

FIG. 5 is an exploded view of a safety syringe of the present invention.

FIG. 6 is a vertical cross sectional view of a safety syringe of the present invention, illustrating the interlocking of the plunger and the needle assembly.

FIG. 7 is a horizontal cross sectional view of a safety syringe of the present invention, illustrating a condition that the drawing mechanism has been inserted into the interlocking receptacle.

FIG. 8 is a horizontal cross sectional view of a safety syringe of the present invention, illustrating a condition that the drawing mechanism has been rotated inside the interlocking receptacle and interlocked with the interlocking mechanism of the needle assembly.

FIG. 9 is a horizontal cross sectional view of a safety syringe of the present invention, illustrating a condition that by further rotating an interlocked plunger the needle assembly is released from orientation.

FIG. 10 is a vertical cross sectional view of a safety syringe illustrating a condition that the needle has been drawn back up into the syringe barrel.

### DETAILED DESCRIPTION OF THE INVENTION

Reference will now be made in detail to the preferred embodiments of the present invention, examples of which are illustrated in the accompanying drawings. Wherever possible, the same reference numbers are used in the drawings and the description to refer to the same or like parts.

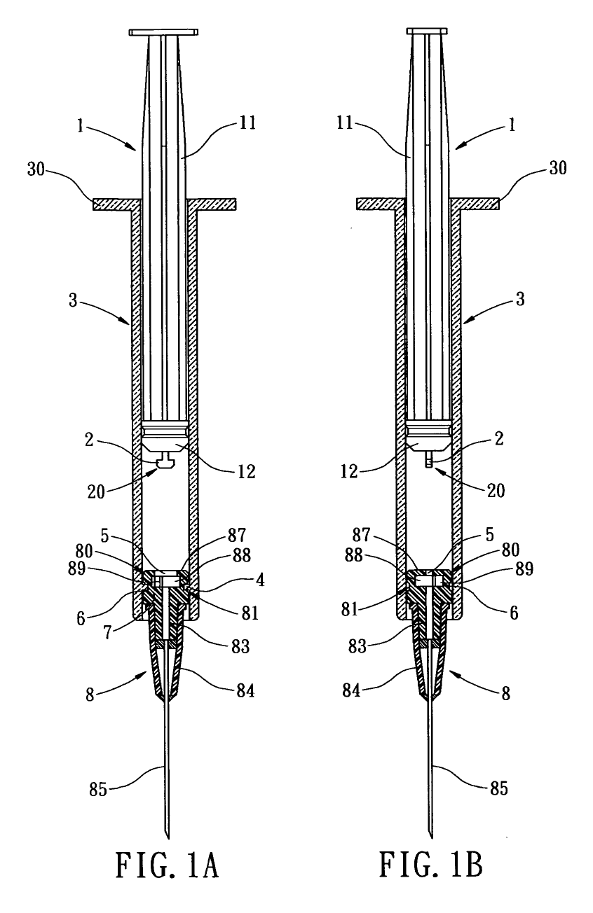

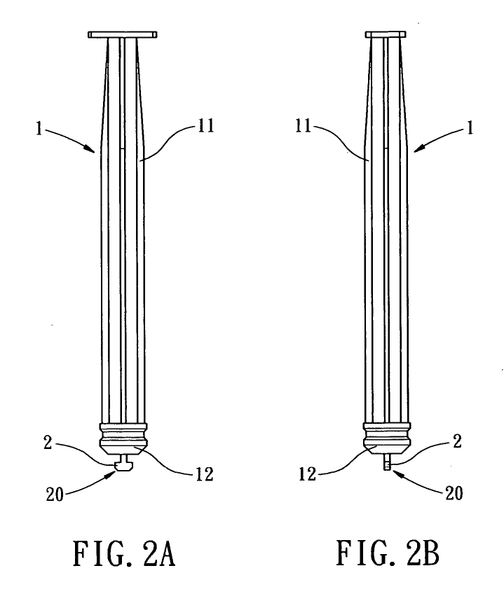

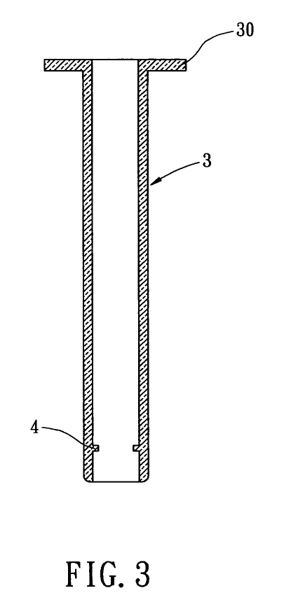

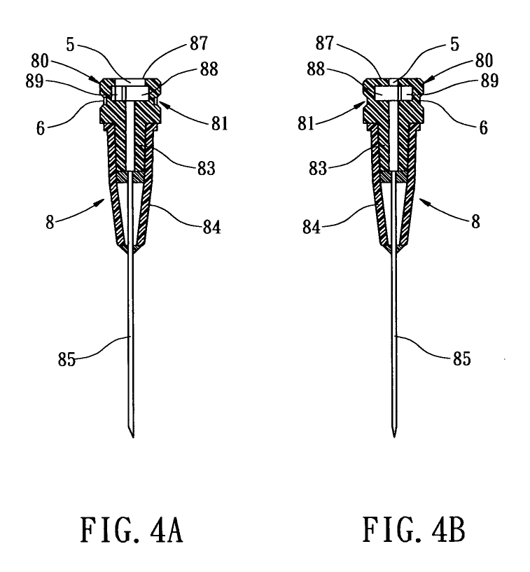

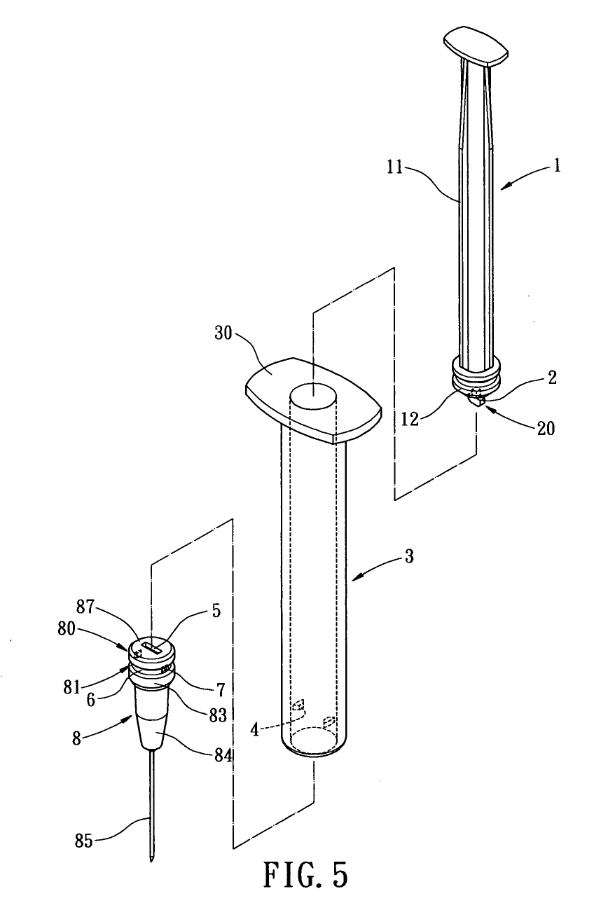

FIG. 1A through FIG. 5 are utilized to describe a preferred embodiment of a safety syringe of the present invention. The safety syringe includes a syringe barrel 3; a needle assembly 8, installed inside the syringe barrel 3 by utilizing an orienting mechanism 81; and a plunger installed inside the syringe barrel 3 which is capable of being pushed or pulled within the syringe barrel 3. The syringe barrel 3 is a tube shaped structure with a hollow interior. The syringe barrel 3 has a syringe flange 30 formed at one end thereof, and a pair of retainer buttons 4 which protrude from the hollow interior at the opposing end thereof. The pair of retainer buttons 4 is disposed in a corresponding manner, and is located in the hollow interior in order to provide the needle assembly 8 for orientation and engagement.

The needle assembly 8 is oriented and installed inside the syringe barrel 3 in correspondence with the pair of retaining buttons 4. The needle assembly 8 includes a needle assembly main body 83; a needle support member 84 assembled onto the needle assembly main body 83; and a needle 85 inserted through, and secured and oriented by, the needle assembly main body 83 and the needle support member 84.

The needle assembly main body 83 has an orienting mechanism 81 installed around the exterior circumference thereof, which corresponds to the pair of retainer buttons 4 and is capable of orienting the needle assembly 8 inside the syringe barrel 3. The orienting mechanism 81 has a hook disconnecting guide notch 6 which is circularly and concavely cut around the exterior circumference of the needle assembly main body 83, within which the pair of retainer buttons 4 can have rotational displacement. A pair of concave openings called retainer button sockets 7 are cut from the concave bottom surface of the hook disconnecting guide notch 6, which correspond with the pair of retainer buttons 4, and which are provided to mutually wedge with the pair of retainer buttons 4 during normal operation, in order to form a combination of the needle assembly 8 and the syringe barrel 3.

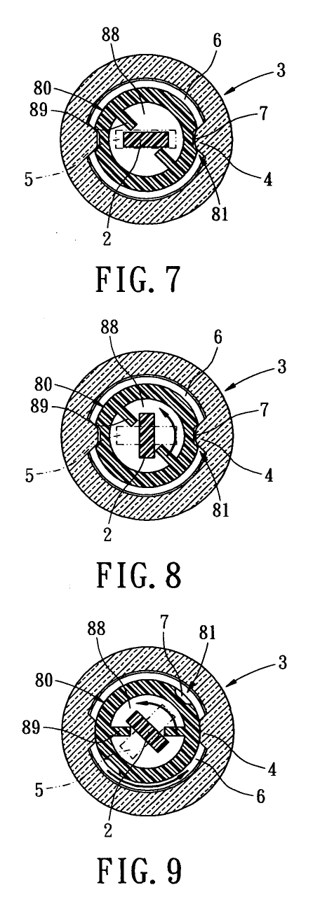

The needle assembly main body 83 has an interlocking mechanism 80 at its bottom end in correspondence with the plunger 1. The interlocking mechanism 80 includes an insert slot 5, which is directly cut from and formed on the rear end wall 87 of the needle assembly main body 83 opposing the plunger 1. The preferred length of the insert slot 5 is one-third the length of the diameter of the rear end wall 87. The insert slot 5 connects to a hollow interlocking receptacle 88. A pair of vertical retaining tabs 89 is formed inside the interlocking receptacle 88. These vertical retaining tabs 89 oppose each other on a diagonal line which has a specific angle with the insert slot 5 as illustrated in FIG. 7. Referring to FIG. 7 through FIG. 9, an anchor shaped reverse hook 2 of the plunger 1 can be inserted through the insert slot 5 into the interlocking receptacle 88, rotated, and combined with the interlocking mechanism 80. The anchor shaped reverse hook 2 is capable of being rotated to push against the vertical retaining tabs 89 in order to drive the needle assembly 8 to rotate for disconnecting the combination of the pair of retainer buttons 4.

The plunger 1 is inserted and installed inside the hollow interior of the syringe barrel 3, and is capable of being pushed forward or drawn backward within the hollow interior of the syringe barrel 3. The plunger 1 includes an plunger main body 11, a piston 12 located at the rear end of the plunger main body 11, and a drawing mechanism 20 installed at the rear end of the piston 12. The drawing mechanism 20 has the anchor shaped reverse hook 2 extended from and formed at the rear end of the piston 12. In one preferred embodiment, the anchor shape reverse hook 2 has a narrow flat structure. The length of the widest portion of the anchor shape reverse hook 2 corresponds with the width of the insert slot 5 of the interlocking mechanism 80. When the plunger 1 is being pushed toward the top-most point, the anchor shape reverse hook 2 is capable of inserting through the insert slot 5, hiding inside the interlocking receptacle 88 of the interlocking mechanism 80 for carrying out the safe retraction of the needle assembly 8 into the syringe barrel 3.

Referring again to FIG. 1A and FIG. 1B, the needle assembly 8 is oriented and secured inside the syringe barrel 3 by the orienting mechanism 81 and the retainer buttons 4. Because the retainer buttons 4 are wedged into the retainer button sockets 7, the needle assembly 8 can resist the reaction force and stay immovable when a medical person pokes the needle into a vial to draw up medication into the syringe barrel 3.

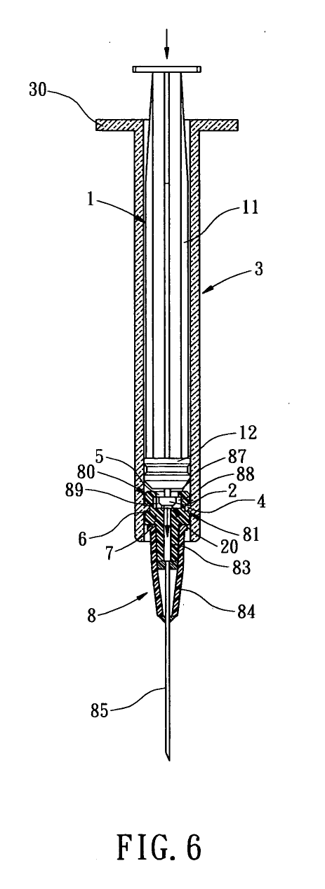

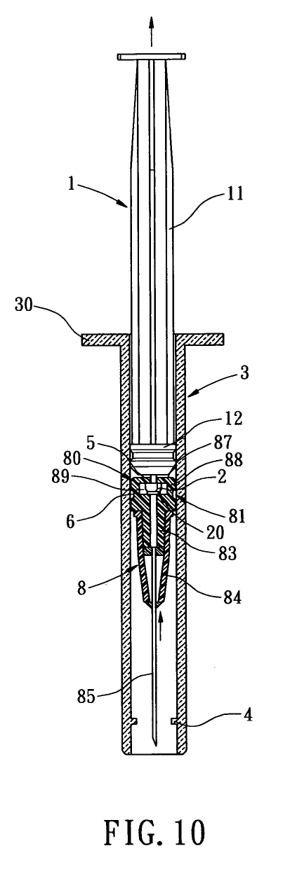

The retraction of the needle assembly 8 back into the syringe barrel 3 is illustrated by FIG. 6 through FIG. 10. After pushing out the medication, the plunger 1 is adjacent to the tip of the syringe barrel 3. The plunger 1 can be further interlocked with the needle assembly 8. The medical person inserts the anchor shaped reverse hook 2 through the insert slot 5 into the interlocking receptacle 88, as illustrated in FIG. 6 and FIG. 7. She further rotates the plunger 1 ninety degrees to hide the anchor shaped reverse hook 2 beneath the rear end wall 87 of the needle assembly 8. The anchor shaped reverse hook 2 will press against the vertical retaining tabs 89, as illustrated in FIG. 8. She still further rotates the plunger 1 approximately fifteen degrees in the same direction. Because the anchor shaped reverse hook 2 presses against the vertical retaining tabs 89, this second rotation will drive the needle assembly 8 to rotate synchronously. The retainer buttons 4 will thereby be disengaged from the retainer button sockets 7 of the needle assembly 8. The needle assembly 8 is therefore released from orientation. Because the retainer buttons 4 have been slid into the hook disconnecting guide notch 6 as illustrated in FIG. 9, the needle assembly 8 is capable of being pulled upward. Further, because the anchor shaped reverse hook 2 is installed beneath the rear end wall 87 of the needle assembly 8, the plunger 1 is interlocked with the needle assembly 8. Pulling the plunger 1 upward will draw the needle assembly 8 into the syringe barrel 3. The contaminated needle is thereby stored inside the syringe barrel 3. Medical personnel or refuse collection personnel will not be pricked or infected by exposed needles during their works.

While an illustrative and presently preferred embodiment of the invention has been described in detail herein, it is to be understood that the inventive concepts may be otherwise variously embodied and employed and that the appended claims are intended to be construed to include such variations except insofar as limited by the prior art.

What is claimed is:

1. A syringe comprising:
a syringe barrel with a retainer buttons formed oppositely on the interior thereof;
a plunger including a drawing mechanism; and
a needle assembly including a needle assembly main body, installed inside the syringe barrel, and oriented by an orienting mechanism which is disposed on the needle assembly main body and correspondingly wedged with the retainer button, the needle assembly main body further having an interlocking mechanism corresponding to the plunger for interlocking with drawing mechanism

2. The syringe of claim 1, wherein the orienting mechanism has a hook disconnecting guide notch formed around the exterior circumference of the needle assembly main body, and a retainer button socket which is a concave opening oppositely formed from a concave bottom surface of the hook disconnecting guide notch to normally wedge with the retainer button.

3. The safety syringe of claim 2, wherein the hook disconnecting guide notch is circularly and concavely cut around the exterior circumference of the needle assembly main body.

4. The safety syringe of claim 1, wherein the interlocking mechanism includes an insert slot, an interlocking receptacle and a pair of vertical retaining tabs, the insert slot is directly formed on the rear end wall of the needle assembly main body corresponding to the drawing mechanism and connected to the hollow interlocking receptacle, and two vertical retaining tabs are formed inside the interlocking receptacle and opposite to each other on a diagonal line which has a specific angle with respect to the insert slot.

5. The safety syringe of claim 4, wherein the width of the insert slot is long enough for the drawing mechanism to insert through in order to interlock with the interlocking mechanism.

6. The safety syringe of claim 1, wherein the drawing mechanism of the plunger has an anchor shaped reverse hook protruding from the rear end of the piston.

7. The safety syringe of claim 2, wherein the drawing mechanism of the plunger is capable of inserting into the interlocking receptacle of the interlocking mechanism, rotating and interlocking with the needle assembly main body, pressing against vertical retaining tabs, driving the needle assembly to rotate synchronically for releasing the needle assembly from orientation, and drawing the needle assembly back into the syringe barrel.
